(19) United States
(12) Patent Application Publication
(10) Pub. No.: US 2020/0238060 A1
(43) Pub. Date: Jul. 30, 2020

(54) DEVICE AND METHOD FOR DRAINING BIOLOGICAL LIQUID AND DETECTING OBSTRUCTIONS

(71) Applicants: Universidad EIA, Envigado (CO); Universidad CES, Medellin (CO)

(72) Inventors: Allen Mejía, Envigado (CO); María Roldán Restrepo, Envigado (CO); Róbinson Alberto Torres Villa, Envigado (CO)

(21) Appl. No.: 16/472,626

(22) PCT Filed: Dec. 19, 2017
(86) PCT No.: PCT/IB2017/058143

§ 371 Date: Jun. 21, 2019

(30) Foreign Application Priority Data
Dec. 23, 2016 (CO) NC2016/0005747

(51) Int. Cl. A61M 27/00; G05D 7/06

(52) U.S. Cl. CPC A61M 27/006; G05D 7/0635; A61M 2205/8206; A61M 2205/3344; A61M 2205/3337; A61M 2205/3334

(57) ABSTRACT

The present invention relates to a device and an associated method for draining a biological liquid and detecting obstructions. The device for controlling the flow of a biological liquid comprises: a first tube; a first pressure sensor connected to the first tube; a second tube; a second pressure sensor connected to the second tube; an electromechanical actuator connected to the first tube and to the second tube; and a control unit connected to the electromechanical actuator, to the first pressure sensor and to the second pressure sensor, wherein the control unit controls the electromechanical actuator and determines the pressure differential between the first pressure sensor and the second pressure sensor. This pressure variation allows obstructions to be determined. The method for draining a biological liquid and detecting obstructions compares the measurements of the pressure of the first sensor with a value entered by a user (set point) to actuate the electromechanical actuator. The method also calculates the pressure difference between the measurements of the first sensor and the second sensor to determine the existence and location of an obstruction.

### FIELD OF THE INVENTION

The present invention refers to draining devices and systems for excess liquid fluids, more specifically those related to liquid flow control, preferably biological liquids, such as, for example, cerebrospinal fluid.

### PRIOR ART DESCRIPTION

Edema is defined as swelling caused by fluid accumulation produced by an imbalance in the level of bodily liquids, when blood vessels pour too much liquid on body tissues or when these liquids are retained in the tissues and do not return to the blood vessels, for example, a cerebral edema. Cerebral edema occurs in the human brain. The human brain is embedded in a liquid, which protects it from bumping into the skull walls. This liquid is called cerebrospinal fluid (CSF). This fluid is generated in the ventricles and travels through various structures in the brain before reaching the subarachnoid space where it is reabsorbed. Whenever there is an increase in the cephalic cerebrospinal fluid content, due to obstructions in the liquid circulation or due to imbalances in its production and reabsorption, a case of hydrocephalus or interstitial edema is established. This disease usually results in increased intracranial pressure and it is commonly treated with a peritoneal-ventricle shunt, which consists of a valve that drains fluid from the ventricles into the peritoneum, where excess fluid is absorbed.

Hydrocephalus is classified according to its cause: obstructive and communicating. In the first case, there is an obstruction in the circulatory pathway of the fluid, so it accumulates in one of the ventricular structures. In the second case, the increase in fluid around the brain is due to an imbalance in the fluid production and reabsorption rate. Both cases belong to a broader classification called common hydrocephalus, which generates a significant increase in intracranial pressure. Along with this classification, a second type of hydrocephalus is established, known as normotensive, in which the increase in fluid increases the cavity volume involved in the fluid circulation, but does not affect the pressure, so a patient with this type of hydrocephalus presents a symptomatology very similar to common hydrocephalus, but maintains a normal intracranial pressure.

The most commonly used treatment for hydrocephalus is the insertion of a peritoneal-ventricle shunt valve (PVS), which is not a cure for the disease, as the damage to the brain tissue remains, but its use keeps pressure under control by draining excess cerebrospinal fluid. There are currently two types of valves in the market, fixed pressure and programmable. In the case of fixed systems, the CSF is only drained from a value that may not be changed at any time, while in programmable valves the specialist may vary this value as often as required by a non-invasive magnetic mechanism. In the latter case, it is necessary to know how the increase in CSF or the change in the patient's intracranial pressure has been in order to reprogram the system.

Unfortunately, due to the poor correlation between clinical signs and intracranial pressure (ICP), the only way to know these changes is to measure them directly. Nowadays, the most outstanding methods for intracranial monitoring are: cranial ultrasound, computerized axial tomography, intraventricular catheter or external ventricular drainage, subarachnoid screw, epidural monitor, spinal tap (when there are no signs of focality) and intraparenchymatous monitors. Unfortunately, all of them are expensive methods that tend to be subjective, given their application and result depends on the conditions and criteria of the physician and also, mostly invasive, implying a high risk of infection.

US 2016/0101270 A1 discloses the drainage of cerebrospinal fluid through a catheter from the ventricles to the peritoneum. Making use of an actuator, as a final control element, controlling the liquid flow by partially opening and closing the catheter. For this purpose, the actuator pinches the catheter on its outer surface, closing the catheter lumen and restricting flow to the peritoneum.

Such a frequent process of pinching the catheter from its outer surface, generates wear on material, which may result in the catheter rupture or the detachment of particles that would be ready to travel through the torrent of the cerebrospinal fluid. On the contrary, the present invention proposes a system with two catheters, a proximal one connected to one end of the final control element and another distal one connected to the opposite end of the final control element, in order to control the flow by means of a sterile actuator obstructing the passage of cerebrospinal fluid towards the peritoneum from the inside of the system, thus preventing the mechanical drive from having repercussions of wear on the conduits through which the cerebrospinal fluid flows, or contamination thereof.

US 2016/0101270 discloses the use of a differential pressure measurement to estimate an approximate value of intracranial pressure. The system periodically closes and opens the actuator in order to compare the estimated intracranial pressure value with an expected pattern, the result of this comparison is related to the presence of a distal or proximal obstruction. US 2016/010270 A1 proposes the use of rotameters to determine the flow through the system. On the other hand, in the present patent, the detection of obstructions is carried out in the following way: it measures the pressure at the entrance of the device and measures it again at the exit of the same one, when the actuator is open, both pressures must be parallel; in case these are not, an obstruction may be determined.

US 2016/010270 A1 does not take into account location. In the present invention, the obstruction location (distal or proximal) is found according to the sign having the difference between the pressure at the inlet of the device and the pressure at the outlet of the device. The present invention makes use of the pressure difference to calculate the flow through the system.

U.S. Pat. No. 7,309,330B2 provides a drainage method and system including a ventricular catheter, a drainage catheter, and a displacement pump that may function to drain cerebrospinal fluid from the ventricles of a patient's brain. The pump may include, for example, a diaphragm pump, a piston pump, a rotor pump, a peristaltic pump or a screw pump. In an invention embodiment, the pump drive is controlled based at least in part on the monitoring of symptoms or changes in the patient's symptoms, the time of day, the patient's circadian rhythms, the appearance of various sleep patterns, the cardiac cycle, an accelerometer monitoring the patient, the patient's intracranial pressure, a siphon control device, or some combination thereof Another invention embodiment provides a drainage system including a ventricular catheter, a drainage catheter, a siphon control device, a bypass, a bypass valve, and a positive displacement pump, which actively operates draining the CSF from the ventricles of a patient's brain. The siphon control device actuates the bypass valve sending flow through the bypass, when an overdrain in the ventricles is detected and through the distal catheter, when the overdrain condition has been mitigated.

It is important to point out that the presence of a bypass may generate a counterflow of cerebrospinal fluid towards the brain, which is very dangerous for the patient's health. Although U.S. Pat. No. 7,309,330B2 develops a method for overdrainage, it does not report obstructions in the catheters.

In conclusion, there is no standalone device in prior art, which meets the comprehensive features in the present invention.

### BRIEF DESCRIPTION OF THE FIGURES

FIG. 1 shows an embodiment of invention with the constitutive parts of the device.

FIG. 2 shows a device mode for draining liquid coupled with an induction coil.

FIG. 3 shows the pinching mechanism (4a) coupled with the electromechanical actuator (6) and the first pressure sensor (5a). Similarly, the fixation mechanism (4b) coupled with the electromechanical actuator (6) and the second pressure sensor (5b) is observed.

FIG. 4 shows the operation of the electromechanical actuator preferred mode (6) a bistable valve.

The FIG. 5 shows an invention embodiment where the device is connected by radio frequency, has a USB module and a display mechanism, which in this case is a PC screen.

FIG. 6 shows a flow chart of the method for draining cerebrospinal fluid and for detecting obstructions.

FIG. 7 shows the flow diagram of an invention embodiment of the invention method by calculating the flow of the cerebrospinal fluid.

FIG. 8 shows a Wheatstone bridge connected to the pressure sensors.

FIG. 9 shows a sensor signal conditioning circuit.

FIG. 10 shows an H-bridge associated with the electromechanical actuator.

FIG. 11 shows a load circuit of the external coil.

FIG. 12 shows a load circuit of the internal coil.

### BRIEF DESCRIPTION OF THE INVENTION

The present invention corresponds to a device and an associated method for liquid drainage and detection of obstructions. The device for controlling liquid flow comprises a first conduit; a first pressure sensor connected to the first conduit; a second conduit; a second pressure sensor connected to the second conduit; a electromechanical actuator connected to the first conduit and to the second conduit and a control unit connected to the electromechanical actuator, to the first sensor and to the second pressure sensor; where the control unit controls the electromechanical actuator and determines the pressure differential between the first pressure sensor and the second pressure sensor. This variation in pressure makes it possible to determine obstructions.

The method for liquid drainage and clogging detection compares the pressure measurements of the first sensor with a value entered by the user (set point) to actuate the electromechanical actuator. In the same way, the method calculates the pressure difference between the measurements of the first sensor and the second sensor, thereby determining the existence of obstruction and location thereof.

### DETAILED DESCRIPTION

This invention corresponds to a device and a method for controlling liquid flow and detecting obstructions.

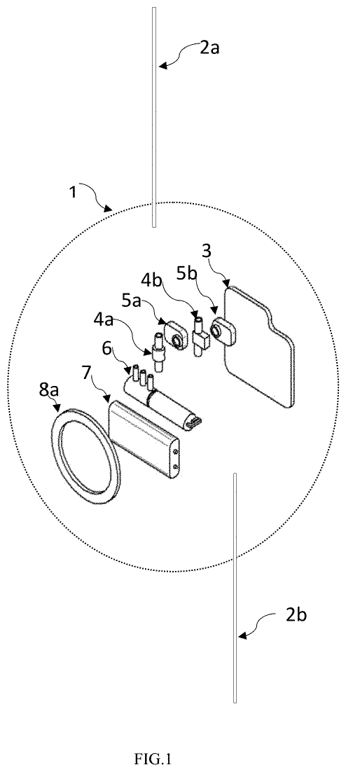

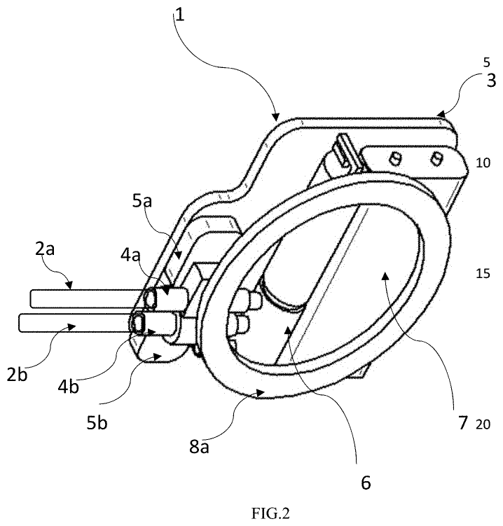

Referring to FIG. 1 and FIG. 2, a device (1) is shown to control the liquid flow, which comprises:
a first conduit (2a) with a proximal end in an area of excess fluid;
a first pressure sensor (5a) connected to the first conduit (2a) at its distal end;
a second conduit (2b) with a distal end in a waste area;
a second pressure sensor (5b) connected to the second conduit (2b) at its proximal end;
an electromechanical actuator (6) connected to the first conduit (2a) at its distal end and to the second conduit (2b) at its proximal end; and
a control unit (3) connected to the electromechanical actuator (6), to the first sensor (5a) and to the second pressure sensor (5b);

where the control unit (3) actuates the electromechanical actuator (6), according to the pressure signals recorded by the first sensor (5a) and compared to a set point value predetermined by the user.

where, the control unit (3) calculates the differential of the pressure signals sent by the first pressure sensor (5a) and by the second pressure sensor (5b) thus determining the presence of obstructions in the first conduit (2a) and/or the second conduit (2b).

For interpretation purposes, the liquid of the present invention may be for example organic, such as biological liquids (e.g. cerebrospinal liquid, lymph, urine, sputum, amniotic liquid, saliva, mother's milk, blood, among others), petroleum, petroleum derivatives, oil, sugars among others or inorganic such as water, ammonia, hydrochloric acid among others depending on their composition.

In an invention embodiment, the first conduit (2a) is housed in the area of excess fluid at its proximal end. The first pressure sensor (5a) connected to the first conduit (2a) at its distal end measures the pressure of the excess liquid zone. The second conduit (2b) will carry the liquid to the waste area. The second pressure sensor (5b) connected to the second conduit (2b) measures the liquid pressure going to the waste area. The liquid comes from the proximal end of the first conduit (2a) and passes through the entire first conduit (2a) to its distal end where it is connected to the electromechanical actuator (6). The electromechanical actuator (6) controls the liquid flow from the first conduit (2a) to the second conduit (2b). The decision whether or not allowing the liquid to pass through the electromechanical actuator (6) is taken by the control unit (3). The control unit (3) decides to actuate the electromechanical actuator (6) taking into account the pressure signals sent by the first sensor (5a), the second sensor (5b) and a pressure set point. From the above, if the liquid passes through the electromechanical actuator (6) it will enter the proximal end of the second conduit (2b) it will flow through the entire conduit (2b) to the distal end of the second conduit (2b) where it will reach the waste area.

In the presence of obstructions and having its location in the first conduit (2a) and/or second conduit (2b). If there is obstruction in the first conduit (2a) its removal will be decided. Similarly, if obstruction is present, only the second conduit (2b) will be removed. In the event both the first conduit (2a) and the second conduit (2b) are obstructed, both will be removed simultaneously.

The first conduit (2a) and the second conduit (2b) may be a tubular organ (e.g. esophagus, intestines, stomach, etc.), or a synthetic conduit (e.g. hoses, ducts, etc.).

In the preferred embodiment, the first conduit (2a) and the second conduit (2b) are made of a biocompatible material. For the purpose of interpreting this invention, biocompatible material means material that complies with ISO-10993: “Biological Evaluation of Medical Devices”. Some materials are: Polyaryletherketone (PAEK), polyetheretherketone (PEEK), high density polyethylene (HDPE), ultra-high molecular weight polyethylene (UHMWPE), polymethylmethacrylate (PMMA), (PSU), Polyetherimide (PEI), Polyphenylsulfone (PPSU), and Polyphenylene Sulfide (PPS), Commercially Pure Titanium (ASTM F67), Titanium Alloys ASTM B265 (standard specification of titanium and titanium in strip, sheet and plate form), Stainless Steel AISI 316L, Medical Grade Acrylonitrile Butadiene Styrene (ABS), polycarbonate, polyamide, polyester, polyvinyl chloride (PVC), polypropylene, polystyrene, polyglycolic acid, polydoxanone, Polyglecaprone, Catgut, Polyglactin 910, silicone and acrylic materials.

Referring to FIG. 2, a first fixation mechanism (4a) coupled to the first conduit (2a) at its distal end, to the first pressure sensor (5a) and to the electromechanical actuator (6) and a second fixation mechanism (4b) coupled to the second conduit (2b) at its proximal end, to the second pressure sensor (5b) and to the electromechanical actuator (6).

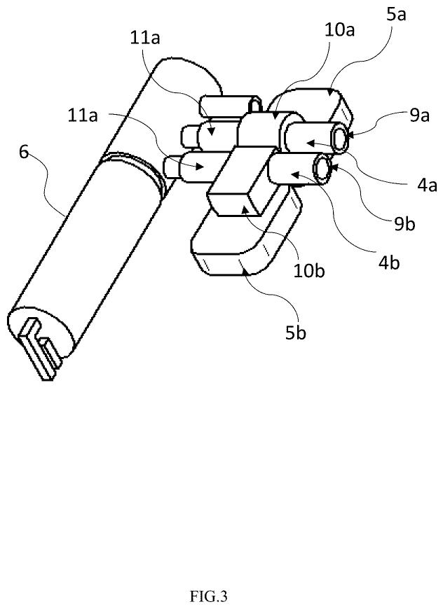

Referring to FIG. 3, the fixation mechanism (4a) comprises a first coupling nozzle (9a), a second coupling nozzle (11a) and a central coupling bracket (10a). Similarly, the fixation mechanism (4b) comprises a first coupling nozzle (9b), a second coupling nozzle (11b) and a central coupling bracket (10b).

The liquid from the excess liquid zone flows down the first conduit (2a) to the first fixation mechanism (4a). The locking mechanism (4a) is attached to the first conduit (2a) by means of the first coupling nozzle (9a) so the liquid flows without any leakage. Then, the liquid is monitored a first time by the first sensor (5a) which is connected to the fixation mechanism (4a) by means of the central coupling bracket (10a) and finally reaches the electromechanical actuator (6) through the second coupling nozzle (11a). The fixation mechanism (4b) comprises a first coupling nozzle (9b), a second coupling nozzle (11b) and a central coupling bracket (10b). The liquid that goes to the distal area, goes down the second conduit (2b) from the second fixation mechanism (4b). The fixation mechanism (4b) is connected to the second conduit (2b) by means of the first coupling nozzle (9b) so the liquid flows without any leakage. The liquid is monitored by the second sensor (5b) which is connected to the fixation mechanism (4b) by means of the central coupling bracket (10b) and finally reaches the electromechanical actuator (6) through the second coupling nozzle (11b).

Referring to FIG. 3, the device (1) characterized by the electromechanical actuator (6) is selected from the group comprised of ball valve, axial flow valve, compression valve, y-shaped valve, butterfly valve, solenoid valve, bistable solenoid valve and combinations thereof.

In an invention embodiment, the electromechanical actuator system (6) is ON/OFF because it is considered that the pressure changes being measured are not sufficiently considerable to require a means of regulation.

The electromechanical actuator (6) needs to be closed or opened when the control unit (3) so decides.

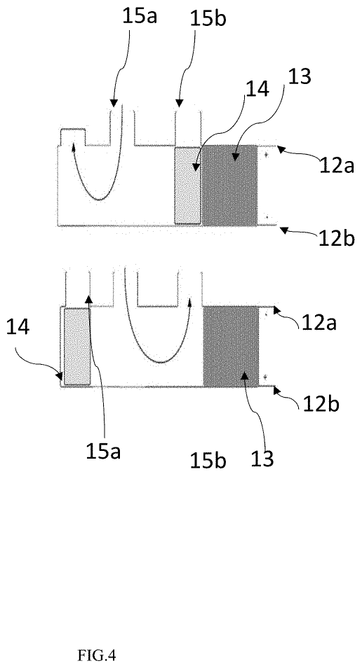

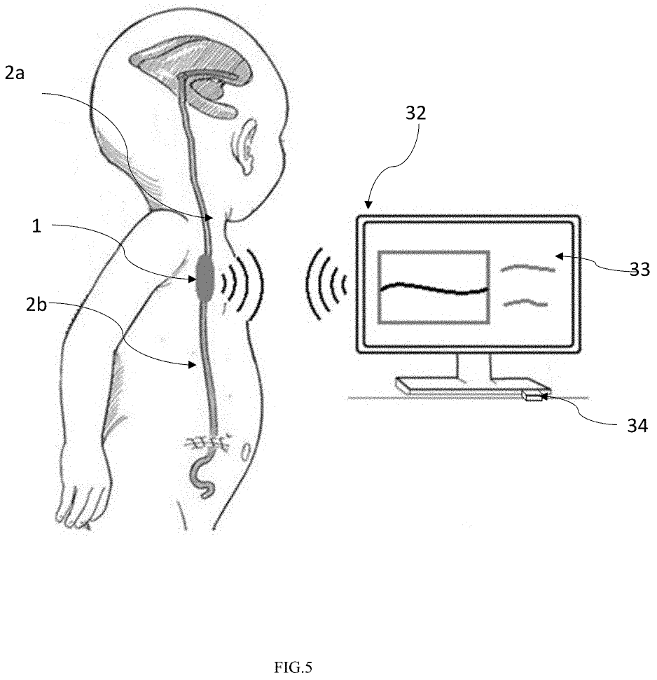

Referring to FIG. 4, in the preferred invention embodiment, the electromechanical actuator (6) is a bistable solenoid valve. The electromechanical actuator (6) consists of an inlet nozzle (15a) and an outlet nozzle (15b). The inlet nozzle (15a) is operationally connected by means of the fixation medium (4a) to the distal end of the first conduit (2a). In this way, the inlet nozzle (15a) is where the liquid coming from the first conduit (2a) enters the electromechanical actuator (6). On the other hand, the outlet nozzle (15a) is operationally connected by means of the fixation medium (4b) to the proximal end of the second conduit (2b). In this way, the outlet nozzle (15a) is where the liquid coming from the electromechanical actuator (6) flows into the second conduit (2b). The operation of the electromechanical actuator (6) consists of an internal coil (13) with a first current input (12a) and a second current input (12b) which exchanges the polarity of its field according to the direction in which the electric current flows. Therefore, if the current goes in one direction from the first current input (12a) to the second current input (12b), the generated field is positive and attracts the negatively charged moving metal (14) inside the valve to close the flow path from the inlet nozzle (15a) to the outlet nozzle (15b). On the other hand, if the current direction is from the first current input (12b) to the second current input (12a), the generated field is negative so the moving metal (14) is repelled and the liquid flow path from the inlet nozzle (15a) to the outlet nozzle (15b) is opened.

The use of an electromechanical actuator (6), a first conduit (2a) and a second conduit (2b) is because it improves the ease of change in case of obstructions, since if the obstruction is found in the first conduit (2a) only the first conduit (2a) will be replaced. Similarly, if the obstruction is found in the second conduit (2b) only the second conduit (2b) will be replaced. In contrast, the use of a single conduit with a pinching or clamping actuator may cause the conduit to wear out, resulting in two undesirable consequences. First of all, the conduit when the worn out material coming from this wearing may get into the drained liquid, which may cause damage to the user. Similarly, the wearing may cause the conduit to require excessive changes, so the user will have many surgical procedures if only one conduit is used.

In an invention embodiment, the current direction of the electromechanical actuator power supply (6) may be changed by means of a device selected from the group between H-bridges with transistors, H-bridge with mosfet, H-bridge with bjt, H-bridge with ibgt, H-bridge with relays, inverters and combinations thereof.

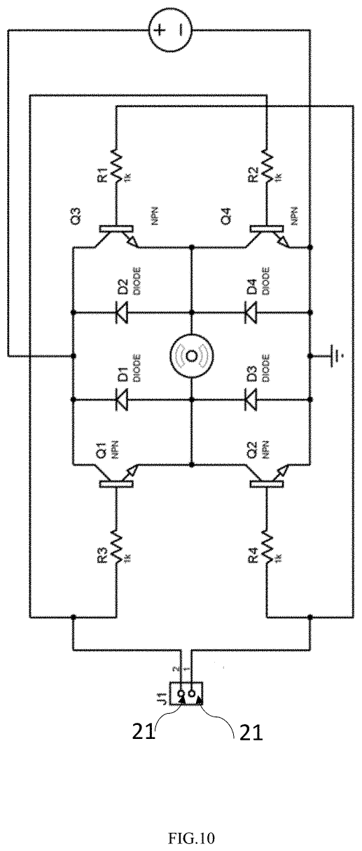

Referring to FIG. 10, in the preferred invention embodiment, an bjt H-bridge is used, the control unit (3) generates a current that is sent via inputs (35a) and (35b) to control the H-bridge. The H-bridge is used to generate changes in the current direction by means of an electronic control governed by the control unit (3). For the H-bridge to be active, the control unit (3) must generate a current in the electromechanical actuator (6). In such a way that if no liquid draining is required, the control unit (3) generates a current in the coil (13) by placing the pin (35a) high and the pin (35b) low, so no liquid flows between the inlet nozzle (15a) and the outlet nozzle (15b), while in the event the pressure in the excess liquid zone is greater than the set point value predetermined by the user, a current is induced in the coil by placing the pin (35a) under and the pin (35b) high, in such a way the valve is opened to drain the liquid, through the nozzles (15a) and (15b), until the pressure stabilizes. The bistable solenoid valve only requires a pulse of 10 ms to change state, so each pin switch only lasts at high intervals of approximately 10 ms required by the actuator.

In the present invention, it must be understood that the user refers to the person who is using the device (1), or a third party with knowledge of its use.

The device (1) is characterized by an anti-flow system connected to the second conduit (2b) and to the electromechanical actuator (6). The anti-reflux system ensures the liquid will always flow from the excess liquid zone to the waste zone and not in the opposite direction, regardless of the user's position with respect to the device (1).

In an invention embodiment, the anti-flow system may be selected from the group comprised of swing flap valve, spring valve, piston valve, ball check valve and combinations thereof. In the preferred invention embodiment, the anti-reflux system is a ball check valve. The ball check valve is selected because it is the one industrially used in application of small sizes.

The device (1) is characterized by the fact the first pressure sensor (5a) and the second pressure sensor (5b) are selected from the group using an operation principle ranging from strain gauges, optical fibers, bourbon tube, diaphragm, piston type, bellows, manometer, capacitive, piezoelectric, optical, surface acoustic waves, bridgman gauge and combinations thereof.

In the preferred invention embodiment, the first sensor (5a) and the second sensor (5b) employ the principle of strain gauges. Strain gauges allow the measurement to be made, due to the measurement deformation when a pressure change is generated. The change in pressure results in a different resistive value. The resistive value is amplified through the coupling of the strain gauge to the instrumentation amplification medium.

In an invention embodiment, the device (1) characterized by the first pressure sensor (5a) and the second pressure sensor (5b) are connected to:
an instrumentation differential amplifier;
a low-pass filter connected to the instrumentation differential amplifier;
an amplifier circuit connected to the output of the low-pass filter;
an analog-to-digital converter connected to the low-pass filter output; and
the control unit (3) connected to the output of the analog-to-digital converter.

In an invention embodiment, the low-pass filter filters and amplifies the signal simultaneously. So the amplifier and filter circuit is done in a single stage. That is, the amplifier circuit is inside the low-pass filter.

In an invention embodiment the instrumentation differential amplifier may be selected within the group comprised, which has at its entrance between a Wheatstone bridge, Wien bridge, Hay bridge, Kelvin bridge, Maxwell Carey Foster bridge and combinations thereof, in conjunction with a differential amplifier. In the preferred mode, the instrumentation differential amplifier is a Wheatstone bridge and a differential amplifier.

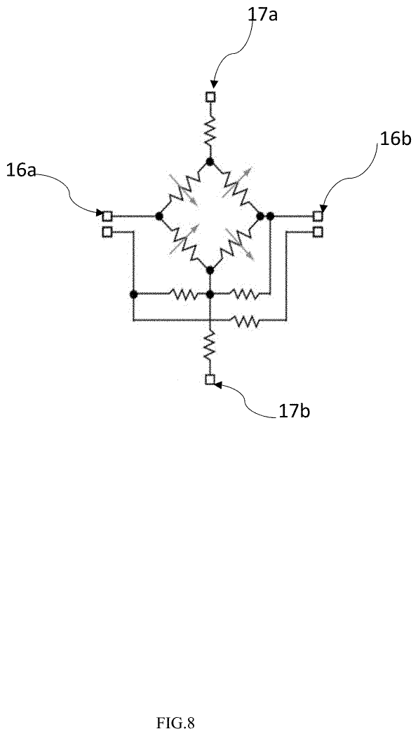

Referring to FIG.8, the first sensor (5a) and the second sensor (5b) will each have the Wheatstone bridge. The Wheatstone bridge consists of 4 pins, two through which it is fed (17a) and (17b), and two others (16a) and (16b) by which the pressure differential of response changes is obtained.

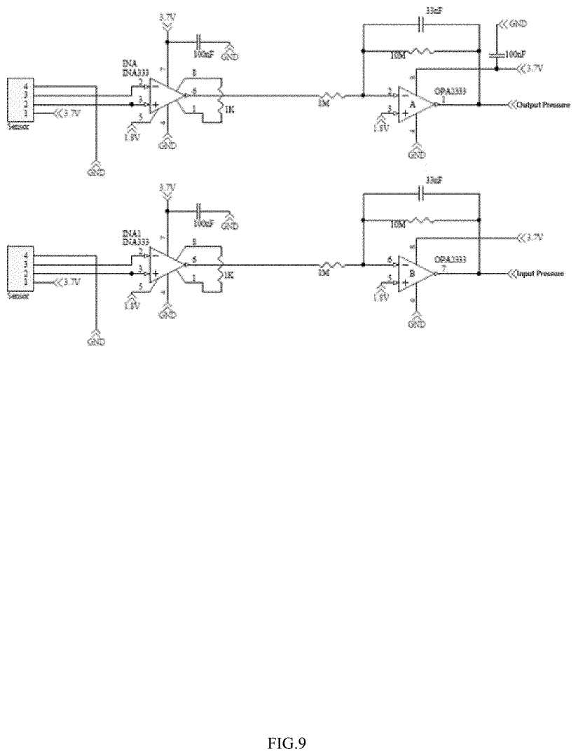

Referring to FIG. 9, in an invention embodiment, the pressure signal conditioning module is connected to the signal detection medium on the Wheatstone bridge pins (16a) and (16b).

The conditioning module may also comprise an amplifier circuit connected to a low-pass filter as follows:
an instrumentation amplifier with a gain, calculated from G=1+(100 kΩ/Gain Resistance), of 11;
an active low-pass filter with a cutoff frequency connected to the output of the instrumentation amplifier; and,
both amplifiers connected to the 3.7V supply with decoupling capacitors.

In an invention embodiment, the low-pass filter has a cutoff frequency calculated from Cutoff Frequency=1/(2*π*Resistance2*Capacitance).

In the preferred invention embodiment, the low-pass filter has a cut-off frequency of 0.5 Hz.

Each of the sensors (5a) and (5b) consists of an independent conditioning module, so both pressure signals enter the control unit (6) to be processed.

The control unit (3) is responsible for controlling the electromechanical actuator and for transmitting and receiving pressure data, finding obstructions. The control unit (3) may be a microcontroller (e.g. 4-bit processor, 8-bit processor, 16-bit processor, 32-bit processor, 64-bit processor, among others).

In the preferred invention embodiment, the control unit (3) comprises a 32-bit processor with 256 kB of flash memory and 16 kB of RAM.

The pressure signals from the first sensor (5a) and the second sensor (5b), previously conditioned by the conditioning module, are recorded via an A/D channel from the analogue to digital converter and a square signal is separated at a stipulated frequency, which is recorded via the control unit (3).

In an invention embodiment, the analog-to-digital converter is integrated in the control unit (3).

In another invention embodiment, the analog-to-digital converter and the control unit (3) are two different elements connected.

When considering each amplified pressure signal, which may be represented by s (t), it is sampled at a time interval T. The analog-to-digital converter provides a discrete signal represented as, x (nT) for n=(1 . . . N), over the NT sampling period of the analog-to-digital converter.

In an invention embodiment, the sampling frequency is in a range of 1 Hz and 5 Hz. In the preferred invention embodiment, the sampling frequency is 1 Hz.

The control unit (3) compares the discrete pressure signals sent by the first sensor (5a) and the second sensor (5b) with each other. The control unit (3) then compares the pressure of the first sensor (5a) with a user-determined set point value. This pressure comparison establishes whether the control unit (3) actuates the electromechanical actuator to allow the liquid flow into the waste area.

The control unit (3) will have a default initial set point value. The set point value may be changed if the user considers it so.

In an invention embodiment, device (1) comprises a battery (7) supplying power to the control unit (3), the electromechanical actuator (6), and the pressure sensors (5a) and (5b). However, it must be understood the unit may be connected to any type of electrical source allowing it to power the device. In an invention embodiment, the battery (7) may be selected among the group comprised between primary batteries, secondary batteries, biodegradable batteries and combinations thereof.

In an invention embodiment the battery (7) is implantable rechargeable and may be selected from the group between nickel-cadmium (Ni—Cd), sealed lead-acid, nickel-metal hydride (NiMH), various types of lithium batteries among which are lithium-polymer (Li-Poly), lithium-ion (Li-Ion), lithium-metal (Li-Metal) and combinations of the above. For the preferred mode, an implantable rechargeable Li-ion battery (7) with a nominal voltage of 3.7V and a capacity of 200 mAh is selected to power the system.

In an invention embodiment, the rechargeable battery (7) may be recharged remotely. Remote recharging may be selected from a group comprised of induction circuit, kinetic energy generation. In an invention embodiment, the rechargeable battery (7) uses an induction circuit to recharge.

In an invention embodiment, the rechargeable battery (7) is connected to:
a voltage regulator circuit (18a);
a first coil (8a) connected to the voltage regulator circuit;
a second induction coil (8b) magnetically coupled to the first coil (8a); and
an induction circuit (18b) connected to the second coil;
   where the induction circuit (18b) generates a variable current in the second coil (8b) inducing a variable field which induces a current in the first coil (8a), being regulated by the voltage regulating circuit (18a).

In an invention embodiment, the rechargeable battery (7) is implantable and recharged by the induction circuit (18b).

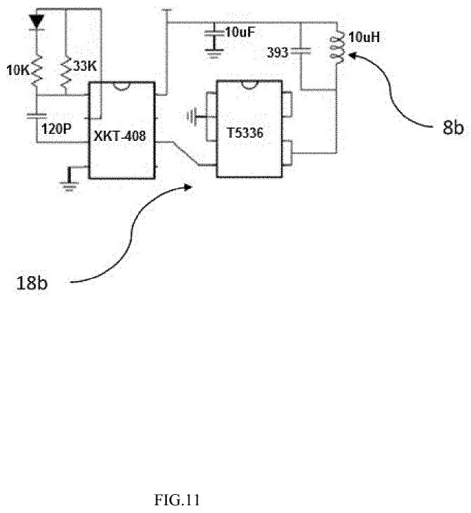

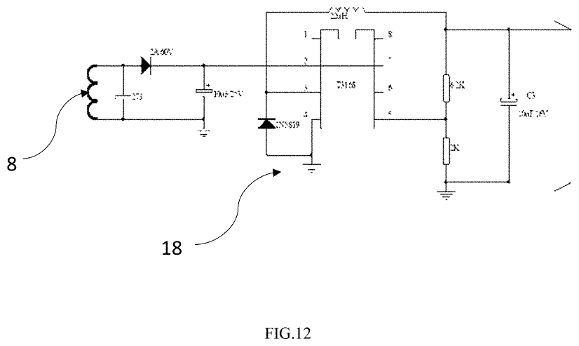

Referring to FIGS.11 and 12, in an invention embodiment, the voltage regulator circuit (18a) connected to the implantable rechargeable battery (7) creates an electric current from the change in the magnetic field created by the induction circuit (18b) outside the patient. Remote recharging is implemented in order to charge the implantable rechargeable battery (7) as many times as necessary, without having to surgically intervene the patient, before the end of the battery life.

By the induction principle, when the induction circuit (18b) is connected to a power supply the field induced in the second coil (8b) is variable, so in the first coil (8a), located in the device (1), an electric current is generated which is used by the voltage regulator circuit (18a) to draw a certain voltage.

In an invention embodiment, the first coil (8a) will be internal to the user and the second coil (8b) will be external to the user. The first coil (8a) and the second coil (8b) are magnetically connected with the user's skin in the middle.

In an invention embodiment, the voltage output of the voltage regulator circuit (18a) is used by a first operational amplifier to recharge the implantable rechargeable battery (7). Similarly, a rechargeable implantable battery charging pin (7) connected to the first operational amplifier is set low to indicate the rechargeable implantable battery (7) is being charged and is high again when it is fully charged. The information provided by the rechargeable implantable battery charging pin (7) is sent to the user to indicate it is possible to remove the second coil (8b) from the user's skin and disconnect it.

When the system is not in recharge mode, the voltage regulator circuit (18a) and the induction circuit (18b) are inactive and only the system is powered by the implantable rechargeable battery (7). The output voltage of the implantable rechargeable battery (7) is subjected to a second operational amplifier. The second operational amplifier raises the output voltage of the rechargeable implantable battery (7), thus obtaining the output voltage required for the operation of the electromechanical actuator (6). The first pressure sensor (5a), the second pressure sensor (5b) and the control unit (3) are supplied with different voltages. Therefore, a first voltage regulator is needed for the first pressure sensor (5a) and the second pressure sensor (5). Similarly, a second voltage regulator for the control unit (3). The first voltage regulator and the second voltage regulator are connected to the output of the second operational amplifier. The presence of voltage regulators is due to the existence of different elements with different supply voltages. In the preferred mode the voltage regulator circuit (18a) uses an integrated T3168, and the induction circuit (18b) a control chip XKT-408A.

In an invention embodiment, device (1) is implantable. The implantable device (1) is characterized by the fact the control unit (3), the electromechanical actuator (6), the first coil (8a), the implantable rechargeable battery (7) are inside a casing (3) of biocompatible material.

In an invention embodiment, the casing material (3) is selected from the group between Polyaryletherketone (PAEK), Polyetheretheretherketone (PEEK), High Density Polyethylene (HDPE), Ultra-high molecular weight polyethylene (UHMWPE), polymethylmethacrylate (PMMA), polysulfones (PSU), polyetherimide (PEI), polyphenylsulfone (PPSU), and polyphenylene sulfide (PPS), Commercially Pure Titanium (ASTM F67), Titanium Alloys ASTM B265 (standard specification of titanium and titanium in strip, sheet and plate form), Stainless Steel AISI 316L, Acrylonitrile Butadiene Styrene (ABS), medical grade, polycarbonate, polyamide, polyester, polyvinyl chloride (PVC), polypropylene, polystyrene, polyglycolic acid, polyoxanone, polyglecaprone, Catgut, Polyglactin 910, acrylic materials. silicone and combinations thereof.

In an invention embodiment the housing material is an implantable liquid silicone coating.

As the device (1) is implantable, it is possible to prevent damage to the components, when in contact with the environment. Similarly, the user of the rechargeable implantable device (1) may have more facilities for its movement.

Referring to FIG. 5, the device (1) where the control unit (3) is connected to a communication means.

In an invention embodiment, the communication means is selected from the group between wired communication, wireless communication and/or combinations thereof.

The communication means may be wireless. In the same way, the wireless communication means may be selected from the group between radio frequency, microwaves, luminous, and combinations thereof.

In the preferred invention embodiment, the communication means is selected from an ultra-low power radio frequency module at 2.4 GHz (34), for Wireless applications. The radio frequency module at 2.4 GHz (34) is used because it has greater coverage, since the attenuation in the air is lower than other bands, for example 5 GHz, compatibility with all current Wi-Fi devices such as tablets, smartphones, consoles, laptops etc., it is recommended to set up long distance links in the band of 2.4 GHz, if the spectrum is not saturated, because the communication goes further (has less air attenuation).

The communication means is in charge of transmitting data between the device (1) and the user, the data transmitted may be those of the pressure, the set point value or the presence of obstruction and its location. The communication means will connect the signals sent by the user with the device (1). Consequently, the user may modify the set point value without having to remove the device (1) from the user.

In an invention embodiment, the device (1) is characterized because the control unit (3) is connected to one or more data display mechanism.

The data visualization is important because it will give the user the opportunity to know how the data varies, in order to know some abnormal behavior.

In an invention embodiment the display mechanism may be selected in the group between Cathode Ray Tube (CRT), Liquid Crystal Display (LCD), Plasma Display Panel (PDP), TFT LCD (Thin Film Transistor), LED display (Light Emitting Diode), OLED (Organic Light-Emitting Diode), AMOLED (Active Matrix OLED), Super AMOLED (Super Active Matrix Organic Light-Emitting Diode) and combinations thereof.

In an invention embodiment, the display mechanism is a LED computer screen (33). In another invention embodiment, the display mechanism is a cellular screen.

The device (1) characterized by the fact the control unit (3) has a wireless communication means and the display mechanism is the cellular LED display. In the cellular LED display case, the user will be able to follow the data thrown by the device (1) and make changes from any place.

In an invention embodiment the device has a USB (Universal Serial Bus) module (34). The USB module (34) is responsible for: receiving data sent from the implantable device to display them in the display mechanism. Moreover, the USB module (34) may contain the communication means and send the data entered by the user to the implantable device. The USB module (34) allows the use of display mechanisms with no wireless communication.

Referring to FIG. 5, in an invention embodiment, the implantable rechargeable device (1) is located in the intra-abdominal region this location is very common in neonates, because no changes have to be made as the neonate grows. Similarly, the location in the intra- abdominal area in the case of adults gives easy access to the device (1) and prevents the fact of shaving the head as it avoids a cranial surgery. The device (1) will be placed in this intra-abdominal region by means of a small incision, the liquid to be drained is cerebrospinal fluid, where the area of excess liquid is the right lateral ventricle, the waste area is the peritoneal area. The first conduit (2a) at its proximal end is in the user's right lateral ventricle and at its distal end is connected to the device (1). On the other hand, the second conduit (2b) at its distal end is in the peritoneal zone and at its proximal end is connected to the device (1). The first conduit (2a) and the second conduit (2b) will be inserted through the same incision through which the device was inserted (1). In the presence of obstructions, the first conduit (2a) and/or the second conduit (2b) may be removed with a simple surgical procedure, without removing the device (1). This generates many advantages in procedure times and user recovery. The device (1) may also be located in any other zone of the torso.

In another invention embodiment the device (1) is connected to an external part of the body, e.g. the user's arm. In the device (1) the exterior has the advantages that if any device component (1) suffers a damage its change may be made without any intervention. Also, the battery charging (7) may be done in a simpler way.

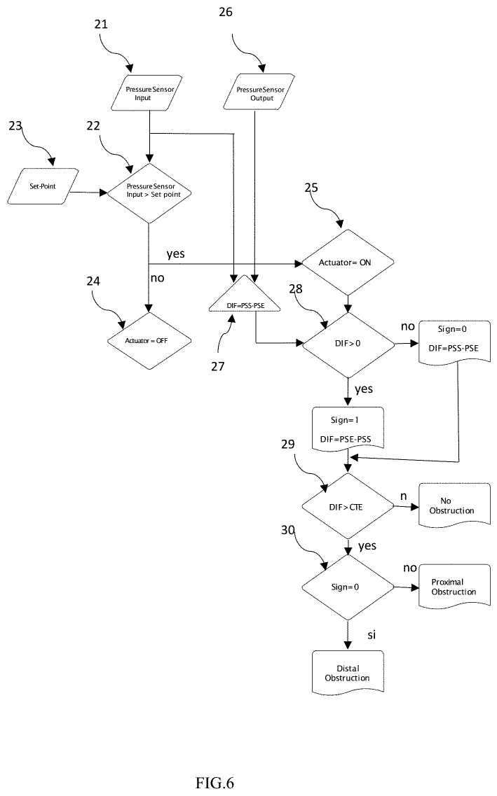

Referring to FIG. 6, a method for draining liquid and detecting obstructions, characterized by the following steps:
a) place the electromechanical actuator (6) between an excess liquid zone and a waste zone to drain liquid;
b) set a default pressure set point (22);
c) measure the pressure in the excess liquid zone (21);
d) actuate the electromechanical actuator (25) to drain liquid when the pressure measurement in the excess liquid zone (21) is above the pressure set point (22) plus one millimeter of mercury; and close the electromechanical actuator (24) when the pressure measurement in the excess liquid zone (21) is equal to or less than the set point (22);

In an invention embodiment, the method for draining liquid and detecting obstructions will be associated with device (1).

In step b, the user establishes a pressure set point (22), which will depend on the characteristics of each user.

In stage c, the pressure measurement in the excess liquid zone (21) of the electromechanical actuator (6) is that delivered by the first pressure sensor (5a).

The control unit (3) in stage d decides whether or not to activate the electromechanical actuator by comparing the pressure measurement in the excess liquid zone (21) measured by the first sensor (5a) with the pressure set point value (22) of stage b.

In an invention embodiment, the control unit (3) uses a control system to compare the pressure measurements selected between the control group of two positions (on-off), control of two positions (on-off) with hysteresis, proportional variable time, proportional, proportional integral, proportional derivative, proportional integral derivative and combinations thereof. In the preferred mode, the control unit (3) uses a two-position (on-off) control system with hysteresis.

The user may also wirelessly send the set point value over the communication means.

The method for draining liquid and finding blockages, also has the following steps:
e) measure the pressure in the waste area (26);
f) determine the pressure differential (27) between the pressure measurement in the excess liquid zone (21) and the pressure in the waste zone (26);
g) compare if the pressure differential is greater than a constant value predetermined by the user (29) then there is obstruction, otherwise there is no obstruction (30);
h) determine the location of the obstruction, if the pressure difference value is greater than zero and greater than the predetermined constant value (29) then the obstruction is in the first conduit (2a), if the value is less than zero then the obstruction is in the second conduit (2b).

In the stage e, the pressure measurement of the waste zone (26) will be taken, which is the one delivered by the second pressure sensor (5b).

The control unit (3) in stage f determines the pressure differential of the pressure in the excess liquid zone (21) and the pressure in the waste zone (26). The control unit (3) in stage g compares whether the pressure differential of the pressure in the excess liquid zone (21) and the pressure in the waste zone (26) is greater than a user-determined constant value (29) depending on which control unit (3) is selected.

This constant is obtained experimentally and depends on the analog digital converter; in the preferred mode the analog-to-digital converter is 10 bits, and the constant has a value of 30.

If the pressure in the excess liquid zone is greater than the constant value set by the user then there is obstruction, otherwise there is no obstruction (30).

The control unit (3) in stage e will determine the location of the obstruction, taking into account if the pressure difference is greater than zero and greater than the predetermined constant value, then the obstruction is in the first conduit (2a) that goes to the excess liquid zone; if the value is less than zero then the obstruction is in the second conduit (2b) that goes to the waste zone.

Referring to FIG. 6 in an invention embodiment, a variable (sign) was used to take values of zero and one. This variable is used by the control unit (3) to compare stages g and e.

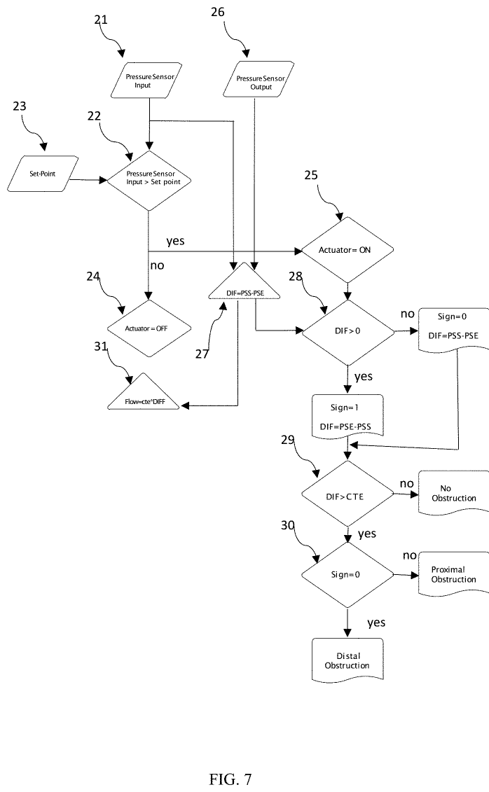

Referring to FIG. 7, where from stage e characterized by calculating the liquid flow in a control unit (3) by multiplying the pressure differential measured in stage e by a constant. The liquid flow is calculated with the following equation:

$$Q = Δ P * CTE$$

$$CTE = \frac{π {r}^{1}}{8 μ L}$$

Where the variables correspond to:

μ=is viscosity

L=is conduit length

r=is conduit radius

The communication means sends the predetermined set point value data, the intracranial pressure, the pressure recording of the last hours, the liquid flow, the absence of obstructions, the presence of obstructions in the first conduit (2a) and the presence of obstructions in the second, conduit (2b) to the display mechanism. In addition, the mechanism displays data for the default set point, liquid flow, lack of obstructions, lack of obstructions in the first conduit (2a) and the presence of obstructions in the second conduit (2b). This data shall be obtained as a result of the method described.

It must be understood the present invention is not limited to the embodiments described and illustrated, because as it will be evident for any skilled artisan, there are possible variations and modifications not departing from the invention scope and spirit, which is only defined by the following claims.

1. A device for controlling the flow of liquid, comprising:
a first conduit (2a) with a proximal end in an area of excess fluid;
a first pressure sensor (5a) connected to the first conduit (2a) at its distal end;
a second conduit (2b) with a distal end;
a second pressure sensor (5b) connected to the second conduit (2b) at its proximal end;
an electromechanical actuator (6) connected to the first conduit (2a) at its distal end and to the second conduit (2b) at its proximal end; and
a control unit (3) connected to the electromechanical actuator (6), to the first sensor (5a) and to the second pressure sensor (5b);
wherein the control unit (3) actuates the electromechanical actuator (6), according to the pressure signals recorded by the first sensor (5a) and compared to a set point value predetermined by the user; where, the control unit (3) calculates the differential of the pressure signals sent by the first pressure sensor (5a) and by the second pressure sensor (5b), thus determining the presence of obstructions in the first conduit (2a) and/or the second conduit (2b).

2. The device of claim 1, comprising a first fixation mechanism (4a) coupled to the first conduit (2a) at its distal end, to the first pressure sensor (5a) and to the electromechanical actuator (6), and a second fixation mechanism (4b) coupled to the second conduit (2b) at its proximal end, to the second pressure sensor (5b) and to the electromechanical actuator (6).

3. The device of claim 1, characterized because the electromechanical actuator (6) is selected from the group comprising a ball valve, axial flow valve, compression valve, y-shaped valve, butterfly valve, solenoid valve, bistable solenoid valve and combinations thereof.

4. The device of claim 1, characterized by an anti-reflux system connected to the second conduit (2b) at its proximal end and to the electromechanical actuator (6).

5. The device of claim 1, characterized because the first pressure sensor (5a) and the second pressure sensor (5b) are selected from the group comprising strain gauges, optical fibers, bourbon tube, diaphragm, piston type, bellows, manometer, capacitive, piezoelectric, optical, surface acoustic waves, bridgman gauge and combinations thereof.

6. The device of claim 1, where the first pressure sensor (5a) and the second pressure sensor (5b) are connected to:
an instrumentation differential amplifier;
a low-pass filter connected to the instrumentation differential amplifier;
an amplifier circuit connected to the output of the low-pass filter;
an analog-to-digital converter connected to the low-pass filter output; and
the control unit (3) connected to the output of the analog-to-digital converter.

7. The device of claim 1, comprising a rechargeable battery (7) supplying power to the control unit (3), the electromechanical actuator (6), and the pressure sensors (5a) and (5b).

8. The device of claim 7, where the rechargeable battery (7) is connected to:
a voltage regulator circuit (18a);
a first coil (8a) connected to the voltage regulator circuit;
a second induction coil (8b) magnetically coupled to the first coil (8a); and
an induction circuit (18b) connected to the second coil;
where the induction circuit (18b) generates a variable current in the second coil (8b) inducing a variable field inducing a current in the first coil (8a), which is regulated by the voltage regulating circuit (18a).

9. The device of claim 1, where the control unit (3) is connected to a communication module.

10. The device of claim 1, characterized by the control unit (3) being connected to a data display mechanism;

11. A method for draining liquid, characterized by the following steps:
a) arrange the electromechanical actuator (6) of claim 1 between an excess liquid zone and a waste zone to drain liquid;
b) set a default pressure set point;
c) measure the pressure in the excess liquid zone (21);
d) actuate the electromechanical actuator (25) to drain liquid when the pressure measurement in the excess liquid zone (21) is above the pressure set point (22) plus one millimeter of mercury; and close the electromechanical actuator (24) when the pressure measurement in the excess liquid zone (21) is equal to or less than the set point (22);

12. The method of claim 11, where after step d, the following steps are performed:
e) measure the pressure in the waste area (26);
f) determine the pressure differential (27) between the pressure measurement in in the excess liquid zone (21) and the pressure in the waste zone (26);
g) compare if the pressure differential is greater than a constant value predetermined by the user (29); if so, then there is obstruction, otherwise there is no obstruction;
h) determine the location of the obstruction: if the value of the pressure difference is greater than zero and greater than the predetermined constant value (29), then the obstruction is in the first conduit (2a); if the value is less than zero, then the obstruction is in the second conduit (2b).

13. The method of claim 1, where from stage e, it is characterized by calculating the liquid flow in a control unit (3) by multiplying the pressure differential measured in step e by a constant.
